(19) United States
(12) Patent Application Publication
(10) Pub. No.: US 2007/0042027 A1
(43) Pub. Date: Feb. 22, 2007

(54) Water soluble anesthetic in an oral patch of hydrophilic gums

(76) Inventor: Jeffrey T. Haley, Mercer Island, WA (US)

Correspondence Address: GRAYBEAL, JACKSON, HALEY LLP, 155 - 108TH AVENUE NE, SUITE 350, BELLEVUE, WA, 98004-5901, US

(21) Appl. No.: 11/492,416

(22) Filed: Jul. 24, 2006

Related U.S. Application Data
(60) Provisional application No. 60/710,432, filed on Aug. 22, 2005.
(60) Provisional application No. 60/795,637, filed on Apr. 26, 2006.

(51) Int. Cl. A61F 13/02

(52) U.S. Cl. 424/448

(57) ABSTRACT

An oral patch for treating toothaches and oral lesions, including mouth ulcers (canker sores/aphthous ulcers) with water soluble topical anesthetics such as dyclonine hydrochloride. In one embodiment, the patch is made by depositing blobs of wet mixture including anesthetic, muco-adhesive hydrocolloids such as collagen, and bingers onto flat sheets. The blobs on the sheets are then dried with air convection, causing the blobs to shrink in height, forming tapered discs with rounded edges. A coating is sprayed onto one side to prevent release of the anesthetic on that side. The base sheets serve as packaging and a lid sheet may be adhered to the base sheet, such as by heat sealing with a hot platen pressing both sheets against an anvil. The platen or the anvil (or both) has recesses that prevent the discs from being pressed during sealing. The sealed package is then cut to size, such as by die cutting.

This application claims priority from U.S. 60/710,432 filed Aug. 22, 2005 and from U.S. 60/795,637 filed Apr. 26, 2006.

### BACKGROUND

It is known that numbing anesthetics are effective for treating pain from toothache and mouth lesions, including ulcers, including aphthous ulcers. To treat such pain, anesthetics such as benzocaine and lidocaine hydrochloride have been delivered in the mouth with liquids, gels, pastes, adherent pressed tablet oral patches and bi-layer non-hydrophilic polymer patches which require that the surface be dry before they are applied. By use of hydrophilic gums to make a patch, the patch will adhere to wet surfaces inside the mouth without first drying them and water soluble molecules that are not too large will diffuse through the wet patch. Benzocaine works poorly in a patch of hydrophilic gums because it is not water soluble and will not diffuse through the patch when the patch is wet, being released only by erosion of the patch. It is not known to put numbing anesthetics in an oral adhesive patch made by depositing blobs of a wet hydrophilic paste and then drying the blobs, or by heating and molding a meltable hydrophilic gum containing the anesthetic. Nor is it known to use dyclonine hydrochloride anesthetic in an oral patch of hydrophilic gums formed by any means.

As used herein, the word “patch” does not include preparations that move about in the mouth rather than adhering in one place, such as cough drops or throat lozenges. Nor does it include preparations that do not hold together as a single item when held in the mouth such as preparations of powder, liquid, paste, viscous liquid gel, or a tablet or troche that crumbles into a powder or paste when chewed or placed in saliva. Conversely, it does include the oral patch made of hydrophilic gums commercially designated with the trademark “Oramelts” disclosed by the same inventor in U.S. patent application Ser. No. 10/287,843 filed Nov. 5, 2002, and U.S. patent application Ser. No. 11/157,054 filed Jun. 20, 2005, each of which is incorporated herein by this reference, as well as other ways of making oral patches from hydrophilic gums discussed below.

### SUMMARY OF THE INVENTION

In one aspect, the invention is an oral patch made predominantly of hydrophilic gums into which is mixed a water soluble anesthetic such as dyclonine hydrochloride or lidocaine hydrochloride or any other water soluble salt of any anesthetic. The hydrophilic gums comprise long chain molecules (polymers) that form a network or matrix. Because the network molecules are hydrophilic, the relatively small water molecules will diffuse through the matrix and water soluble molecules of a size larger than water but much smaller than the polymer molecules, such as the water soluble anesthetics, when dissolved in water, will also diffuse through the matrix. An anesthetic of moderate water solubility such as dyclonine hydrochloride, is preferred because it will linger longer in the human tissues into which it is absorbed rather than passing quickly through the tissues.

In another aspect, the invention is an oral patch containing an anesthetic that is coated on one side with a material that prevents the anesthetic from being released on that side, to minimize exposure of anesthetic to tissues that do not need numbing. The coating material may also be non-muco-adhesive so that the patch does not stick to a surface opposite the tissue to be treated, such as the cheek or lip so that, once placed, the patch will not be pulled away from the treatment site. The coating may be made of a material that resists erosion/dissolution long enough that the anesthetic is mostly released from the other side of the patch before the coating is eroded away. The coating has holes that are too small to pass significant numbers of the anesthetic molecules. Suitable coatings include celluloses, natural latexes and shellacs, and hydrophobic materials such as lipids. Such a material may be applied by spraying and then curing.

In yet another aspect, the invention is an adherent oral patch made by mixing muco-adhesive hydrocolloids and an anesthetic with water into a paste, depositing the paste onto a sheet or mold, and then drying the deposits to form discs. This manufacturing method produces a superior shape for such a patch over other methods because the resulting disc is tapered to a thin edge rather than having a thick or square edge.

The oral patch made of hydrophilic gums will adhere to teeth, gums, cheek, lips, or tongue without the user first drying saliva from the tissue. The user merely places a dry oral patch in his or her mouth on soft, wet tissue or places a moist patch on soft or hard tissue and presses it in the desired location for 10 to 60 seconds. It will adhere to the tissues that it has been touching without movement for 10 to 60 seconds even though those tissues are wet. This is far easier for people to use than requiring that the tissue first be dried with a towel before the adherent oral patch is placed. If the user wants to use an oral patch in the lip or under the tongue, the oral patch can easily be removed for talking and then easily be replaced without using a towel or a mirror.

### BRIEF DESCRIPTION OF THE DRAWINGS

FIG. 1 shows schematically a process for making the patches.

FIG. 2 shows a side view of an oral patch made by depositing wet blobs.

FIG. 3 shows a side view of the patch after it is dried.

### DETAILED DESCRIPTION

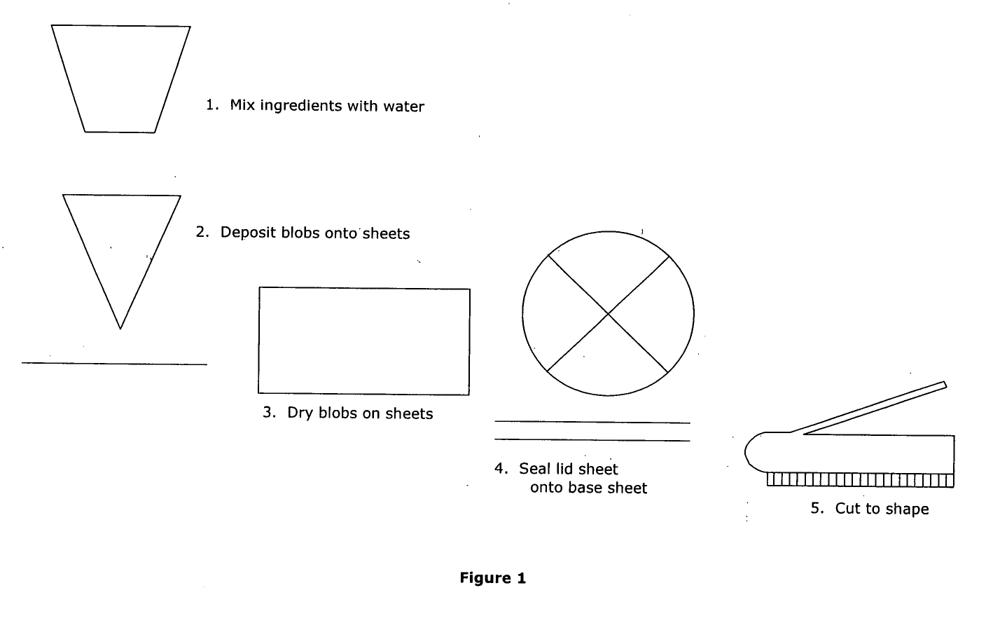

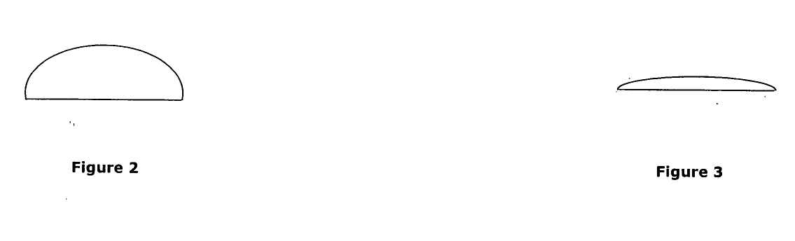

FIGS. 2 and 3 show an adhesive hydrophilic oral patch. It may be provided dry and hard with a shape like FIG. 3 or partially hydrated and soft with a shape like FIG. 2 or between these conditions. It can be prevented from becoming dry and hard by including a non-evaporating liquid as a plastcizer such as natural molecules of glycerin or propylene glycol or synthetic molecules of dibutyl sebacate, triethyl citrate, triacetin, acetylated monoglycerides, dibutyl phthalate, butyl phthalyl butyl glycolate, butyl stearate, diphenyl phthalate, dicyclohexyl phthalate, cresyldiphenyl phosphate, or benzyl phthalate. When soft, the patch has a feel and texture like gummy candies. The preferred shape is a thin lentil which may be nearly flat on one side as shown in FIGS. 2 and 3.

Hydrophilic gum ingredients that have been tested and found to work well include gelatin, carrageenan (preferably kappa), xanthan gum combined with konjac gum, agar, starch, including corn starch and potato starch, carboxymethyl cellulose, hydroxymethyl cellulose, hydroxypropyl cellulose, polyacrylic acid (carbomer), polyvinylpyrrolidone, polyethylene oxide, and carbopol-934. Another useable gum is gum arabic. Other gums similar to those listed, such as locust bean gum which has properties similar to konjac gum, and guar gum should also work. Gelatin is a particularly effective adhesive for inside the mouth.

The hydrophilic gums may include a thermo gel having a melting temperature higher than human mouth temperatures. Preferably, the thermo gel is elasto-plastic, such as formed by carrageenan or a mixture of the hydrogels konjac gum and xanthan gum dissolved in hot water and then cooled to form an elasto-plastic gel. Or it may be comprised of a hydrogel formed of amino acids, such as peptides. Alternatively, the gums may be comprised of a complex carbohydrate, such as cellulose, pectin, maltodextrin, or starch from potato, rice, corn, or wheat.

In a preferred embodiment, the gums include a hydrophilic polymer that liquefies at mouth temperatures and gels to a solid at room temperatures such as gelatin (partially hydrolyzed collagen rendered from animal tissue). Use of such a polymer allows the oral patch to be removed from the mouth and placed on a smooth surface, such as plastic film where it gels. Once the hydrophilic polymer then gels, the oral patch again becomes handleable with the fingers to return it to the mouth without being too sticky to handle or leaving a residue on the fingers or on the plastic film.

An effective quantity of topical anesthetic in each patch is about 3 to 10 milligrams for dyclonine hydrochloride, formulated at about 0.8 to 4% of the non-water ingredients of a 300 to 500 mg oral patch. Lidocaine hydrochloride is not as potent, requiring about 30 to 75 milligrams in each patch, formulated at about 6 to 15 percent of the non-water ingredients in each patch. Bupivacaine is an attractive anesthetic because it is long lasting and has a high ratio of sensation anesthesia to motor anesthesia. Because the anesthetic is concentrated on the toothache or lesion site by the patch, the treatment is effective for the painful site yet does not provide undesirably excessive amounts of the anesthetic to other tissues.

The preferred patch formulation is made by combining the anesthetic with collagen and with other binder ingredients. Collagen, which is the organic molecule that makes up skin and the lining of the mouth (a form of skin), tends to adhere very well to itself, making it glutinous, and therefore adheres very well to the lining of the mouth. An effective and cost effective form of collagen is food grade gelatin which is made from animal skins. If the collagen is bound too tightly by other binder ingredients, the patch will not be adherent enough to stay in place. If the patch is dry and there is too much collagen or other strong adherent such as a combination of carbomer, hydroxpropyl cellulose and polyvinlypyrrolidone, the patch will be too adherent for comfort and may be so adherent that it leaves soft tissues feeling stripped of mucous if it is pulled off.

Testing shows that, if the binder/hydrocolloids are collagen, xanthan gum, konjac gum, and cellulose fiber, effective dry weight formulations have between 0.8% and 15% water soluble anesthetic (dyclonine hydrochloride or lidocaine hydrochloride), between 30% and 75% food grade gelatin (collagen), between 10% and 55% xanthan gum, konjac gum, and cellulose fiber or similar hydrophilic binders, and between 1% and 8% plasticizer such as glycerin or propylene glycol (natural ingredients) or a synthetic plasticizer that is not hydrophobic and, preferably, at least partially water soluble such as triethyl citrate, triacetin, acetylated monoglycerides, or butyl stearate.

The preferred method of manufacturing the patches as shown in FIG. 1 is to use gum drop manufacturing equipment, squirting a hydrated mixture heated above the gel melting temperature through nozzles onto a flat sheet or mold. The deposited blobs are allowed to cool and gel and then the blobs are dried. As they dry, they shrink vertically to form thin edge discs. The patches are preferably dried until the water activity level is lower than 0.8 so that the patches will not grow mold or other organisms. The patches are packaged with a hermetic seal to retard absorption of water moisture from air.

The mixture may be deposited as an array of hot, viscous drops onto a sheet of high temperature plastic or coated paper. Before being deposited, the mixture may be heated to above an activation temperature that is below boiling, and then, after being deposited, the mixture is allowed to cool to form a gel. The drops are allowed to cool and then the sheets of plastic or coated paper with the drops on them are dried in a drying room or convection oven until the water activity level less than 0.8. The product is then packaged still adhered to the plastic or paper and the user pulls it off the plastic or paper. The blobs on the sheets are then dried with air convection, causing the blobs to shrink in height, forming tapered discs with rounded edges.

Instead of forming the patches by deposition, the hydrophilic mixture may be formed into a sheet, to which is applied a hot press that melts and deforms the sheet material to produce patches with a thin, tapered edge and a raised center. By this method, all the material in a sheet can be forced into the body of one of the patches, which may be close-packed rectangles with rounded corners or hexagonally-close-packed circles, leaving no waste trimmings between patches. The sheet may be comprised of two layers so that only one of them is adherent and includes the anesthetic. The heat and pressure can be applied through a layer of flexible packaging material on each side of the patch so that the patches need not touch the hot press surfaces, to minimize contamination risk and problems of the hydrophilic mixture adhering to the press. Suitable flexible materials include any plastic sheet that can withstand the press temperatures, which are typically 180 degrees F. to 220 degrees F., such as nylon or teflon.

The coating may be applied to one side by spraying and then curing. The spray may be applied with one nozzle spraying onto each dried blob with little over spray or the entire sheet of blobs may be sprayed with a uniform coating, in which case the excess spray remains on the sheet as it is sold to the consumer and breaks at the edges of the patches as they are lifted from the sheet. Examples of suitable GRAS-certified materials for a spray coating include but are not limited to celluloses, monoglycerides, triglycerides, waxes such as paraffin, fatty acids, fatty alcohols and mixtures thereof. Sorbitan monostearate (SPAN 60) with hydroxypropyl cellulose (HPC LF) is an effective combination, as well as Aquacoat CPD (cellulose acetate phthalate aqueous dispersion), Marcoat 125 shellac, Surerelease ethylcellulose, and natural rubber latex. Preferred coatings have low water solubility. They may be suspended in any solvent that allows spraying or they may be liquified by heating and then cured by cooling.

Instead of spraying a coating on an exposed surface of the patch after it has been deposited, coating material may be sprayed, printed, rolled, or extruded onto a sheet of plastic onto which blobs of paste are then deposited or pressed, such as 0.020 inch polypropylene. If sprayed or printed, it may be applied only to the spots where the blobs will be deposited in a next pass.

The base sheets serve as packaging and a lid sheet may be adhered to the base sheet, such as by heat sealing with a hot platten pressing both sheets against an anvil. The platen or the anvil (or both) has recesses that prevent the discs from being pressed during sealing. The sealed package is then cut to size, such as by die cutting.

While particular embodiments of the invention have been described above the scope of the invention should not be limited by the above descriptions but rather limited only by the following claims.

I claim:

1. An oral patch made predominantly of hydrophilic gums into which is mixed a water soluble anesthetic such as dyclonine hydrochloride or lidocaine hydrochloride or any other water soluble salt of any anesthetic.

2. The patch of claim 1 further comprising a coating on one side of the patch which coating inhibits passage of molecules of the anesthetic through the coating.

3. The patch of claim 2 wherein the coating is not significantly muco-adhesive.

4. The patch of claim 2 wherein the coating is selected from the group comprising: celluloses, natural latexes and shellacs, and hydrophobic materials such as lipids.

5. An oral patch containing an anesthetic that is coated on one side with a material that prevents the anesthetic from being released on that side, to minimize exposure of anesthetic to tissues that do not need numbing.

6. The patch of claim 5 wherein the coating material is non-adhesive so that the patch does not stick to a surface opposite the tissue to be treated.

7. The patch of claim 5 wherein the coating is made of a material that resists erosion/dissolution long enough that the anesthetic is mostly released from the other side of the patch before the coating is eroded away.

8. The patch of claim 5 wherein the coating has holes that are too small to pass significant numbers of the anesthetic molecules.

9. The patch of claim 5 wherein the coating is selected from a group comprising elluloses, natural latexes and shellacs, and hydrophobic materials such as lipids.

10. The patch of claim 5 wherein the coating is applied by spraying and then curing by one of cooling past a gelation temperature or evaporating away a solvent.

11. The patch of claim 5 wherein the anesthetic is dyclonine hydrochloride.

12. A method of making a numbing adherent oral patch by mixing muco-adhesive hydrocolloids and an anesthetic with a solvent into a paste, depositing the paste in blobs onto a sheet or mold, and then drying the blobs to form discs.

13. The method of claim 12 wherein the resulting disc is tapered to a thin, rounded edge rather than having a thick, square edge.

14. The method of claim 12 further comprising applying a coating to one side of the patch which coating inhibits transmission of molecules of the anesthetic.

15. The method of claim 12 wherein the coating is applied by spraying a squirt onto each dried blob from a nozzle directed at the blob.

16. The method of claim 12 wherein the coating is applied by first applying the coating to a sheet of plastic and then depositing the blobs on the coating.

17. The method of claim 12 wherein the anesthetic is dyclonine hydrochloride.
